(19) United States
(12) Patent Application Publication
(10) Pub. No.: US 2005/0149033 A1
(43) Pub. Date: Jul. 7, 2005

(54) Bioabsorbable fasteners for preparing and securing ligament replacement grafts

(75) Inventors: David A. McGuire, Anchorage, AK (US); Stephen D. Hendricks, Anchorage, AK (US)

Correspondence Address: BROMBERG & SUNSTEIN LLP, 125 SUMMER STREET, BOSTON, MA, 02110-1618, US

(73) Assignee: Dr. David A. McGuire, Anchorage, AK (US)

(21) Appl. No.: 10/971,721

(22) Filed: Oct. 22, 2004

Related U.S. Application Data
(60) Provisional application No. 60/513,652, filed on Oct. 23, 2003.

(51) Int. Cl. A61B 17/56

(52) U.S. Cl. 606/77

(57) ABSTRACT

A bioabsorbable implantable device for replacing sutures in construction of a composite graft in ligament replacement surgery. In certain embodiments the device has a female component and a male component where the female component receives and secures the male component. In other embodiments the components of the device are in the shape of a rivet or a staple. The bioabsorbable implantable devices can be used for securing tendon grafts to bone blocks and for holding together the fibers of the tendon graft when the bone-tendon-bone graft is inserted into a patient during surgery. The bioabsorbable implantable device may also be part of a package for use in surgery. The package includes a sterile container for holding at least a graft that is of a predetermined length and width. The package may also include bone blocks. The package is marked as to the graft size including the width and the length of the graft. The graft and the bone blocks may be autogenous, allogenic or constructed from man-made materials.

### PRIORITY

The present application claims priority from U.S. Provisional Patent Application No. 60/513,652, filed Oct. 23, 2003 having the title “Graft Preparation Table and Bioabsorbable Fasteners for Preparing and Securing Ligament Replacement Grafts,” which is incorporated herein by reference in its entirety.

### TECHNICAL FIELD

The present invention relates generally to surgical devices and procedures used in the field of arthroscopic surgery, particularly to devices for preparation of grafts for reconstruction of the anterior cruciate ligament (ACL) or posterior cruciate ligament (PCL) of the knee, and more particularly to devices for securing connective tissues in any ligament replacement grafts.

### BACKGROUND OF THE INVENTION

When performing anterior cruciate ligament reconstruction a bone tunnel is formed in the tibia and in the femur so that a ligament replacement graft may be inserted and secured therein. A ligament replacement graft may be a semitendonous tendon, a patellar tendon, or other ligament replacement attached to and between a pair of bone blocks which have been sized for close fitting arrangement within one of the femoral and tibial tunnels. The ligament replacement may be from an allogenic (cadaveric) or autogenous (patient) source. One common approach to ACL reconstruction is the use of patellar tendon to form a bone-tendon-bone graft (BTB).

Each type of replacement ligament used in ligament reconstruction techniques has its own set of benefits and complications. Amongst autogenous grafts, complications relate specifically to two areas: graft site morbidity and fixation modality. Complications associated with graft harvest are numerous. The incision used to harvest the patellar tendon is longer than the incision through which arthroscopic surgery is performed. This added length and additional opening into the body contributes additional risk of infection. Since nerve ends are incised during this process, areas of skin on the distal (away from the spine) side of the incision incur nerve anesthesia (dead skin sensation) and proprioceptive loss. Propriceptive loss in the knee can result in gait anomalies, variations in foot alignment, and abnormal wear patterns of the cartilage covered femoral condyles, tibial plateau, or of the menisci.

Another potential complication of BTB harvest is residual pain and discomfort which is separate from the added risk of patellar fracture due to stress risers created during bone plug harvest. Fracture injuries to the patella require significant effort to resolve pathology and related symptoms. These problems of graft harvest are significant enough to have fostered the creation of specialized instrumentation and surgical techniques to minimize both harvest site pain and risk of fracture. Despite advances in autograft bone-plug harvest and defect grafting, pain is a persistent complaint and the risk of patella fracture has not been eliminated.

Another potential complication of common BTB harvest methods is graft fiber relative-width problems that contribute to graft weakness and failure. This occurs when one or both of the harvested bone-plugs is mismatched to the tendon fibers, such that the relative width is less than the desired width which is equivalent to the width of the bone plug. Another way that relative-width error occurs is if the fibers of the tendon are crosscut in order to connect the two closest ends of the misaligned harvested bone plugs.

Graft fiber relative width problems were significant enough to foster the creation of specialized instrumentation and surgical techniques to minimize this problem. Despite advances in Autograft BTB soft-tissue harvest, graft elongation and graft failure still occur and the risk of patellar tendon rupture has not been eliminated.

Another type of autogenous graft used is either or both of the semi-tendinosus, or gracilis (hamstring), the use of which, in addition to the incision complications described previously, includes the risk of harvest site amputation resulting in a graft that is too short to use. Harvest site weakness can produce mobility restrictions and some specific movement incapacity. An additional complication with this type of graft relates to its mode of fixation. Without a bone plug at either end, interference screw fixation, imposed by a headless screw applying lateral force to a boneplug inside the tunnel wall, is not a possibility. Consequently, a staple, screw with washer over a post, or use of a suture attached to an endo button will produce reduction in graft to tunnel wall non-union/non-incorporation from the bungee effect (consistent elongation and contraction under continuous use loading and unloading cycles).

Hamstring harvest and fixation complications were significant enough to foster the creation of specialized instrumentation and surgical techniques to minimize these problems. There has been some limited use of direct interference screw to graft fiber fixation used in lieu of staple, screw with washer over a post or use of a suture attached to an endo button. Despite advances in autograft hamstring harvest, graft elongation, risk of harvest site amputation, non-union/non-incorporation, and graft failure has not been eliminated.

A solution devised to solve fixation problems associated with hamstring use was the incorporation of bone plugs into a looped, double-thick hamstring graft construct. Harvested hamstrings are approximately 2 to 3 times the length and slightly larger than half the diameter required for an ACL substitutes. Consequently, they do not have the same strength as an ACL when compared directly. By folding the hamstring in half, over onto itself, more appropriate lengths and graft strength are obtained. Use of a folded hamstring graft requires some method of fastening the graft to itself. Prior art solutions accomplished this by hand with the use of surgical sutures using various patterns of stitching. Not only is this method of graft construction laborious and tedious, it is complicated by the material itself which is slippery to handle or hold, and the collagen fiber cell matrix resists uniform insertion of the suture needle. These factors add time to the operation, the graft preparation portion of which is but one part.

During the surgery, various means are implemented to reduce risk to the patient while balancing the needs of the surgeon to accomplish the procedures. During ACL reconstruction, there are two specific elements of risk that are typically associated with the surgery. The patient is anesthetized by the use of general anesthesia for reflex and pain control, and a tourniquet is applied to the leg to control bleeding in the surgical region. Generalized goals during the development of arthroscopic instrumentation and techniques for ACL reconstruction has been to reduce the time element of the procedure while improving the efficacy of the technique. By reducing the time element of the procedure, risks associated with general anesthesia and tourniquet use are reduced, and the safety of the procedure for the patient is improved.

One such time element area that remains to be improved is with graft preparation. Problems arise because of the difficulty of running needles through bone both in terms of the mechanics and the accuracy of the procedure, necessitating pre-drilling of suture holes. More prosaically, needles break, physicians run the risk of needle punctures to themselves and to other sites of the patient, and because of its consistency, the ligament replacement often slips out of place before it can be secured. In addition, the sutures remain in the patient indefinitely.

### SUMMARY OF THE INVENTION

A bioabsorbable implantable device for replacing sutures in construction of a composite graft in ligament replacement surgery is disclosed. The device has a female component and a male component. In one embodiment the components of the device are in the shape of a rivet. In another embodiment, the components of the device are in the shape of a staple. In yet another embodiment, the female component may contain unidirectional teeth on an interior surface and the male component may contain opposing unidirectional teeth on an external surface. In such an embodiment, the device forms a circle when the male component is inserted into the female component. The inner arc of the circle can contain protruding spikes for affixing to the patient. In order to increase the diameter of the circle two such devices may be joined together, wherein the male component of a first device is inserted into the female component of the second device.

The implantable device may be composed of a plurality of bioabsorbable materials. In some embodiments, the bioabsorbable device contains one of a group of compounds comprising polydextrolactic acid, polylevolactic acid, pologlycolides, and polydioxanone.

The bioabsorbable device may include a securing device. The securing device may include a first and second arms connected pivotally to one another. Each arm has a distal and a proximal end that define a recess. The distal end of the second arm further comprises a cannulated cylinder wherein the cannulated cylinder is attached along the recess side of the second arm such that the cylinder is parallel to the longitudinal axis of the second arm. The cannulated cylinder also has unidirectional teeth along the inner surface of the cylinder. The securing device also includes an expandable and contractible loop connected to the first and second arms. The loop includes a linear flexible band having a first ringed end and a second non-ringed end and an inside and an outside surface. The outside surface has unidirectional teeth on the second non-ringed end. There is a rigid connection between the first ringed end and the outer surface of the flexible band such that the plane of the ring is essentially perpendicular to the tangent of the flexible band at the connection point. The loop also includes a lariat-like conformation such that the second non-ringed end is threaded through the first ringed end such that the inside surface of the second end is juxtaposed to the outside surface of the first end within the ring. There is a fixed connection between the first ringed end of the circular loop and the distal end of the first arm such that the first ringed end is connected to the recess-side of the first arm and essentially perpendicular to the longitudinal axis of the first arm. The securing device also includes a movable connection between the non-ringed end of the circular loop and the distal end of the second arm wherein the non-ringed end of the circular loop is threaded through the cannulated cylinder at the distal end of the second arm such that the unidirectional teeth on the outside surface of the non-ringed end of the circular loop are capable of being engaged by the opposing unidirectional teeth within the cannulated cylinder at the distal end of the second arm as the recess between the two arms is decreased. The securing device also includes a mechanism for expanding the dimension of the circular loop after it has been contracted.

In yet another embodiment, the bioabsorbable device is a surgical clamp. The clamp includes a pair of opposing clamping members for engaging tissue. A first arm of the clamp is attached at its distal end to one clamping member and a second arm is attached at its distal end to the opposite clamping member. The first and second arms are pivotably connected to one another for variably adjusting a distance between the respective clamping members. The clamping members have mortises complementary to bioabsorbable graft fastener shapes along the inner surface of the clamping members.

In one embodiment, each of the clamping members is arcuate in shape. In another embodiment, the clamping members are parallel to each other. The mortises of the clamp may be complementary to a circlage-type graft fastener. In another embodiment, the mortises are complementary to a rivet-shaped graft fastener. In yet another embodiment, the mortises are complementary to a stapler-shaped graft fastener. The surgical clamp may include a locking mechanism for securely maintaining an engagement between the clamping members. In certain embodiments one clamping member is sized to partially receive the other clamping member.

The bioabsorbable device may be inserted using an implantable device insertion station. The station may include a tensioning track, a pair of blocks for inserting bioabsorbable implantable devices which are separated along the tensioning track where each block has a chamber for accommodating a bone block and openings for bioabsorbable implantable devices on opposite sides of the block sufficiently large to expose the tendons and accommodate the implantable devices. The implantable device openings of the opposite sides are thus being aligned with one another with the chamber therebetween. The station also includes restraints for holding the implantable device blocks an adjustable distance apart from one another along the tensioning track.

Certain embodiments of the invention may also include an instrument for perforating a tendon/bone complex of a prepared composite graft. The instrument includes a drill and a grafting table. The grafting table includes a device for immobilizing the composite graft wherein the two parallel opposite tendon/bone complexes are exposed, and a guidance device for guiding the placement of the drill along the exposed tendon/bone complex of the composite graft wherein the graft may be placed through one of a plurality of channels. The channels being sized to accommodate different widths and types of grafts.

In another embodiment, the bioabsorbable fastener may be part of a preformed package containing either sized components for constructing a bone tendon bone graft or a preconstructed graft for use in reconstructive surgery. The package includes a sterile container that holds the bone plugs, ligament, and bioabsorbable fasteners. The sterile container may be sectioned and may include sterilized fluid for preserving the ligament and/or the bone plugs. In some embodiments the packaging may be hermetically sealed. The package may have separated allogenic graft components or the ligament or bone plug composition may be formed from man-made materials. The packaging also includes markings that indicate sizing of the ligament and of the bone plugs. More specifically, the sizing may indicate the width and length of the ligament.

In one embodiment, the graft may be made in the following manner. A bone block drill guide having a central column and first and second parallel cylindrical columns intersecting opposite side of the central column equidistant from the center of the of the center column is placed over a bone block. The first and second cylindrical columns each have a center and the centers of the central column and the first and second columns all are aligned. The bone block is drilled to create first and second parallel grooves in opposite sides of the bone block. A ligament replacement is attached to the bone block and looped about the bone block along the first and second grooves. The ligament replacement is then tensioned appropriately. The ligament replacement is attached while tensioned to the bone block using a bioabsorbable implantable device.

In another embodiment of the method, the method further requires inserting the first and second bone plugs along with the at least one ligament replacement each into an implantable device insertion block. The bone plugs and the at least one ligament replacement are accommodated in a compatible chamber on each implantable device insertion block and the implantable device openings are aligned on opposite sides of one another with the chamber therebetween. A stop pin is inserted into each of the implantable device insertion blocks so as to keep each bone plug within its respective implantable device insertion block. Simultaneous pressure is applied to opposite sides of the bone plugs at the exposed surfaces of the opposing bioabsorbable implantable device components, thereby locking the ratchet teeth of each component together to achieve the desired clamping force for the implantable device and securing the at least one ligament replacement to the first and second bone plug.

In various embodiments of the method, the bioabsorbable implantable device is shaped like a rivet or a staple. In other embodiments the bioabsorbable device completes a circle when the male component is inserted into the female component. The method may employ any of the described bioabsorbable fastening devices.

The bioabsorbable implantable device may be used to secure ligaments to the bone plugs or for holding the ligament strands together. In the method the ligaments and bone plugs may be made from autogenous, allogenic or man-made materials. In one embodiment, the graft is prepared prior to surgery as a pre-fabricated graft.

The invention may also be embodied as a package for use in reconstructive surgery. The package includes a sterile container for holding at least a graft that is of a predetermined length and width. The packaging is marked as to the graft size including the width and the length. The graft may be an autogenous, allogenic or man-made tendon. The package also includes one or more bioabsorbable implantable devices for use in constructing a bone tendon bone graft. The graft may reside with sterilized fluid within the sterile container and the container may be hermetically sealed. In certain embodiments, the package may also include a pair of bone blocks. The graft may include marking indicating the location for attachment of the bone blocks. The bone blocks may be predrilled having a cavity for inserting the tendon there through. The bone blocks may also be pre-drilled such that the bone blocks include a grove for the tendon to sit in.

### BRIEF DESCRIPTION OF THE DRAWINGS

The foregoing features of the invention will be more readily understood by reference to the following detailed description, taken with reference to the accompanying drawings, in which:

FIG. 1 shows a bone block and tendon graft for insertion into human having tendon or ligament replacement surgery such as ACL surgery;

FIGS. 2A-2D represent examples of composite graft fasteners in accordance with specific embodiments of the present invention. FIG. 2A represents a rivet configuration for a graft fastener; FIG. 2B represents a staple configuration for a graft-fastener; FIG. 2C represents a circlage configuration for a graft fastener; and FIG. 2D represents a U-shaped or semi-circle configuration for a graft fastener.

FIGS. 3A-3D represent examples of clamping devices for securing graft fasteners having the staple or rivet configurations to tissue, in accordance with specific embodiments of the present invention. FIG. 3A represents a basic clamping device having a wing nut-type clamp closure; FIG. 3B represents a basic clamping device having a rotatable sleeve-type clamp closure; FIG. 3C represents a wing-nut-type clamp having a complementary mortise on the inner surface of each clamping member specific for a rivet-type graft fastener; and FIG. 3D represents a rotatable sleeve-type clamp having a complementary mortise on the inner surface of each clamping member specific for a staple-type graft fastener.

FIG. 4 illustrates several variations of circlage-type clamps in accordance with specific embodiments of the present invention, for use with a graft fastener having either a circular configuration or a “U”-shaped or semi-circle configuration. FIG. 4A shows a basic arcuate-type clamp with two combined semi-circle graft fasteners within the arms of the clamp having a composite graft enclosed within the graft fastener; FIG. 4B represents a detail of the clamping members of an arcuate-type clamp showing the complementary mortise along the inner surfaces of the arcuate arms specific for either a circle- or semi-circle-configured graft fastener; FIG. 4C represents an arcuate-type clamp with a circle-configured graft fastener placed within the arms of the clamp, the graft fastener encircling a composite graft; FIG. 4D represents another variation of a circlage-type clamp having a circle-configured graft fastener with a composite graft within.

FIGS. 5A and 5B show miscellaneous views of composite grafts and clamping devices in accordance with specific embodiments of the present invention.

FIG. 6A is an isomeric view of a bone plug made with the present invention showing how rivet-shaped implantable devices can be inserted to secure tendons and/or ligament replacement tissue to the bone plug.

FIG. 6B is a side view of the bone plug, and rivet-shaped implantable devices, of FIG. 6A.

FIG. 6C is an isomeric view of the bone plug showing how staple-shaped devices are inserted.

FIG. 6D is a side view of the bone plug showing the groove for the tendon and the rivet holes.

FIG. 7A is an isomeric view of a bone plug made with the present invention showing how staple-shaped implantable devices can be inserted to secure tendons and/or ligament replacement tissue to the bone plug.

FIG. 7B is a cross-sectional view of FIG. 7A taken along line 3-3.

FIG. 8A shows an end view of an implantable device block for use on the graft preparation table of FIG. 9.

FIG. 8B is a side view of an implantable device block such as that of FIG. 8A showing rivet-shaped holes for insertion of rivet-shaped implantable devices.

FIG. 8C is a side view of an implantable devices block such as that of FIG. 8A showing staple-shaped holes for insertion of staple-shaped implantable devices.

FIG. 9 shows a representation of a graft preparation table.

FIG. 10A shows a side view of a bone block drill guide.

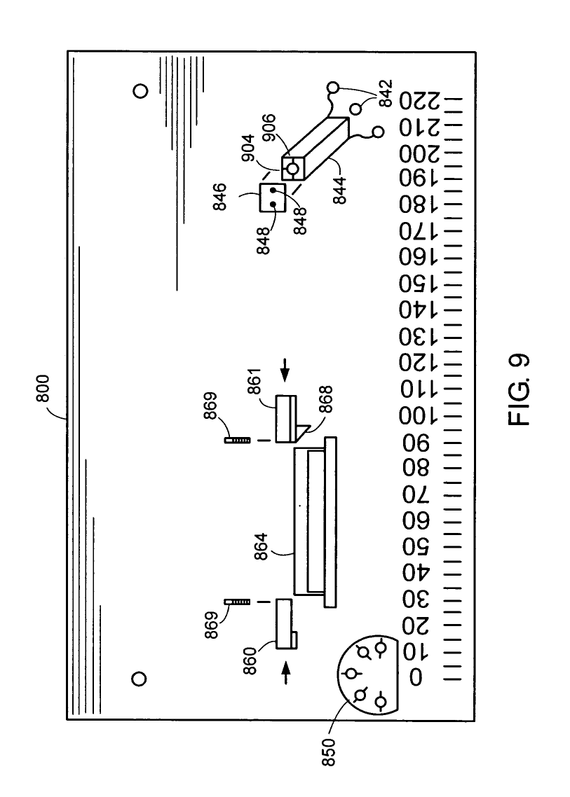

FIG. 10B shows a plan view of the bone block drill guide of FIG. 10A.

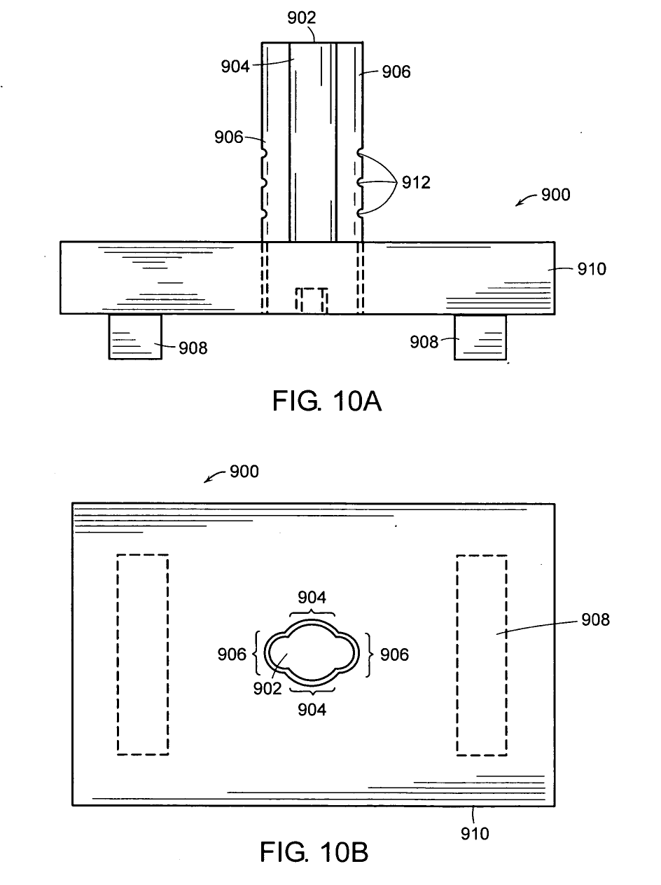

### DETAILED DESCRIPTION OF SPECIFIC EMBODIMENTS

Definitions. As used in this description and the accompanying claims, the following terms shall have the meanings indicated, unless the context otherwise requires: The term “tendon” shall refer to a soft tissue and shall be synonymous with the term ligament.

The composite grafts described utilize bone cores (plugs) taken from the patient or provided from cadaveric donors, and these plugs are combined with semi-tendinosus or other tendons taken from the patient or provided from cadaveric donors. These combined component composite grafts can be entirely autogenous, entirely allogenic, or a combination of each type resulting in an alloautograft. This construct combines the best aspects of both types of materials. For example, if a coring reamer is used to create the tunnels, the resultant bone core may be used as part of a combined alloautocomposite graft along with some autogenous ligament or tendon. The value associated with this combination relates to both types of materials. Allogenic ligaments or tendons may be selected for their size or number for relative strength considerations. Triple strand or quadruple strand tendons may be used in lieu of double strand configurations to result in strength increases commensurate with the increase in relative thickness of the 3 or 4 multiply stranded over the double strand configurations. The value of the autogenous bone plug as non-dead tissue, is that graft incorporation and remodeling are accelerated via the revascularization process. This process is accelerated by the proximity of live bone to live bone such that earlier bilateral revascularization is facilitated by earlier sclerotic margin formation between the autogenous bone plug and the tunnel walls.

The use of this type of composite alloautograft configuration is limited to reconstructions requiring tunnels that surgeons choose to use coring reamers to create. In instances where fully fluted reamers are used to drill the tunnels, the resultant bone debris is unsuitable for the bone end of a composite graft construct. Under these circumstances an entirely allogenic composite graft may be comprised of a bone cores and ligaments or tendons to pre-specified dimensions for a given patient as required. Furthermore, they may be prefabricated as kits to be assembled in the operating room by surgical staff just prior to or during the procedure. They may also be prefabricated and prepackaged completely at a site specifically suited for this purpose rendering them ready to insert after they are rehydrated or thawed and pretensioned.

Such an approach alleviates some of the problems and risks associated with securing ligament replacement to bone, but not all. Although held in place by tensioning blocks, the suturing step is a tedious and slow one, so the ligament replacement could still slip out of place during the procedure. Also, the bone plugs must still be sutured to the ligament replacement tissue using traditional suture materials. Although a graft table provides pre-aligned holes for guidance of sutures, the procedure still involves needles, and so, the risk of inadvertent punctures is still an issue. An additional difficulty is the uniformity of the interface between the graft component materials and the suture materials and associated knots and suture loop configurations and techniques. The resultant outcome variations attributable to intra or inter surgeon differences combined with inter or intra material differences may produce patient outcome complications specific to these variations. A method by which these variations can be minimized is with the use of bioabsorbable fasteners that are of specific types for various applications of material component fastening. The result of the use of these fasteners is the secure integration of composite materials into a composite graft system that will withstand insertion, fixation, integration, and graft remodeling.

Use of these devices to construct composite grafts serves to decrease risk to patients by reducing the tourniquet and anesthesia time typically added during graft preparation. For composite autograft constructs, it permits the combined use of materials, bone cores and ligaments, to be prepared more uniformly and in less time. Multiple source, allogenic, autogenous, or man-made, composite grafts constructed with these fastening devices can utilize otherwise discarded materials such as cored autogenous bone, or biologically created ligaments such as autogenous cells harvested and grown, or man-made components such as synthetic bone replacement products, to create various combinations of composite grafts entirely independent of any harvest and subsequent complications while also reducing tourniquet and anesthesia time. Entirely allogenic composite grafts can be prepared completely independent of the operating room and can be made ready for assembly or be pre-assembled. These pre-prepared or prefabricated kits only requiring assembly by use of the fasteners or only require hydration, or thawing, and pre-tensioning respectively. Not only does use of these devices help the patient by reducing tourniquet and anesthesia time, but the increased uniformity and customization capacity to match individual patient graft dimension and strength requirements can significantly improve patient outcome efficacy while also being less dependent on the variations and complications associated with graft preparation and harvest instrumentation and techniques.

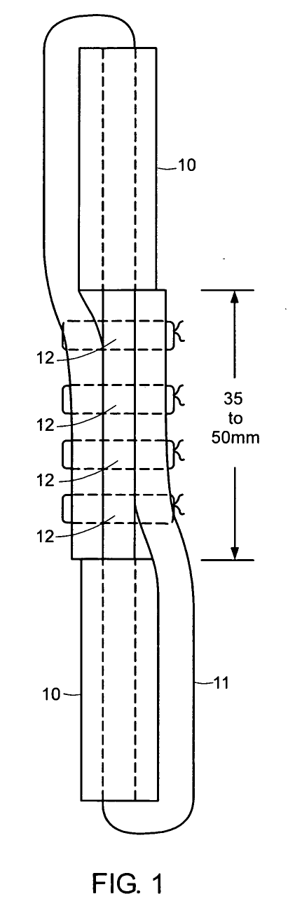

FIG. 1 shows a bone block 10 and tendon graft 11 for insertion into human having ligament replacement surgery such as an ACL surgery. The replacement tendon 11 used in creating the graft may be a pre-packaged tendon. If the tendon is an allogenic or autogenic, the tendon is harvested and stored in sterilized packaging prior to use. Such pre-packaged tendons or ligaments may also be an artificial tendon/ligament. Similar to the allogenic or autogenic soft tissue graft, the artificial tendon/ligament would be wrapped in sterilized packaging. The packaging may be constructed from materials typically used to store sterile medical equipment and may be hermetically sealable. Such pre-packaging allows a surgeon to have pre-sized grafts to be sutured and affixed in a more timely fashion than having to harvest the graft during surgery. Further, the pre-packaged grafts reduce the trauma that the patient undergoes during the harvesting overcoming some of the problems with the prior art. Such pre-packaged tendons are sized by both length and width dimensions which are provided on the exterior of the sterilized packaging. This sizing allows the surgeon to merely measure the patient and select an appropriately sized tendon. The tendon may also have markings indicating where sutures or bioabsorbable implantable device may be used for attachment to a bone block.

The replacement tendon is attached to a bone block 10 at each end of the tendon 11 using a fastener 12, which as discussed below is a bioabsorbable fastener. The bone blocks and bioabsorbable fasteners may also be part of a pre-packaged kit, so that a doctor can quickly select a kit for his intended purpose without the need for extracting bone plugs from the patient, thereby decreasing the length of the surgery. As shown in FIG. 1, the tendon is attached in a figure eight pattern wherein the middle portion of the replacement tendon is constrained by bio-absorbable fasteners 12 or sutures.

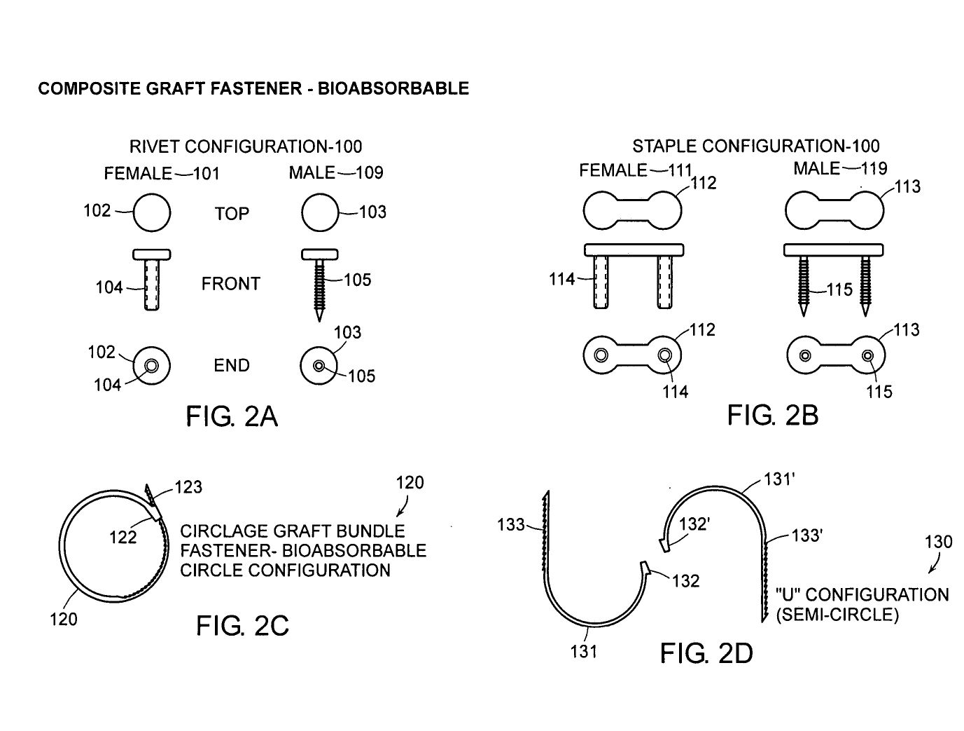

Referring now to FIG. 2A, a rivet-shaped graft fastener 100 in accordance with a specific embodiment of the present invention has a female component 101 and a male component 109, for securing tissue in a composite graft. Female component 101 has a top 102 and a hollow sheath 104 and male component 109 has a top 103 and a grooved or toothed shaft 105. Upon insertion of shaft 105 into sheath 104, a secure union is formed for securing tissue in a graft.

Similarly, in FIG. 2B, a staple-shaped graft fastener 110 in accordance with a specific embodiment of the present invention has a female component 111 and a male component 119 for securing tissue in a composite graft. Female component 111 has a top 112 and two hollow sheaths 114, and male component 119 has a top 113 and two grooved or toothed shafts 115. Upon insertion of shafts 115 into sheaths 114 a similarly secure union is formed for securing tissue in a graft.

Alternatively, a circle-shaped graft fastener 120 as shown in FIG. 2C, in accordance with a specific embodiment of the present invention, may be used for securing tissue in a composite graft. Circle fastener 120 has a hollow female component 122 at a first end having unidirectional teeth within, and a male component 123 at a second end having opposing unidirectional teeth. Upon insertion of male component 123 into female component 122, a secure union is formed between components 122 and 123 and a circle is completed for enclosing and securing tissue in a graft.

Similarly, a semi-circle or U-shaped graft fastener 130 as seen in FIG. 2D, in accordance with another specific embodiment of the present invention, may be used for securing tissue in a composite graft. Two U-shaped graft fastener components 131 and 131′ are required to form one complete graft fastener 130 in this example. Each U-shaped fastener 131 and 131′ has a hollow female component 132 and 132′ having unidirectional teeth within at one end, and a male component 133 and 133′ having opposing unidirectional teeth at the other end. Upon insertion of each of two male components 133 from two identical U-shaped fasteners 130 and 130′ into each of two corresponding female components 132 of the same two identical U-shaped fasteners, one completed graft fastener is formed for enclosing and securing tissue in a graft.

Note that the above graft fasteners are all designed to be bioabsorbable implantable devices such that when used in conjunction with reconstructive surgery they do not have to be removed at some later date but can be left in the body to degrade over time. Preferred bioabsorbable materials that would be suitable for such devices include polylactic acids such as polydextrolactic acid, polylevolactic acid, polyglycolites and polydioxanoe. The bioabsorbable materials that are listed are only illustrative and other bioabsorbable materials and their stereo isomers along with any combination thereof may be used. For example, two bioabsorbable materials may be combined in order to achieve the proper time for absorption such as polydextrolactic acid which has a fast absorption time may be combined with polylevolactic acid which has a slower absorption time.

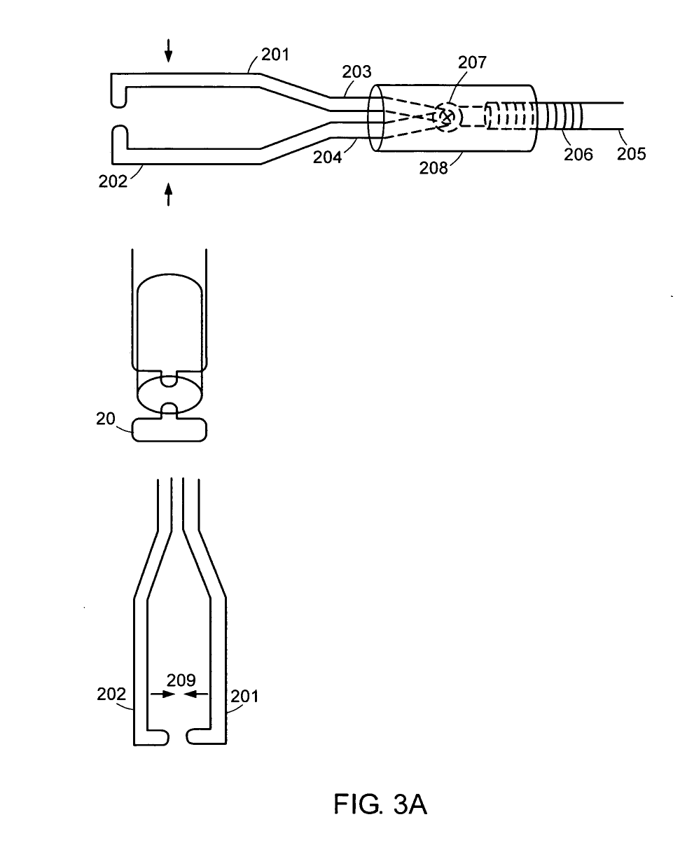

Referring now to FIG. 3A, a general two-armed clamp 200 is shown for securing bioabsorbable implantable devices such as rivet-shaped device 100 and staple-shaped device 110. Clamp members 201 and 202 are attached to arms 203 and 204, respectively, which in turn are connected by pivot point 207 to a central shaft 205. Clamp 200 may be closed or opened using either a sleeve-type clamping mechanism 208 or a wing-nut-type clamping mechanism 210. If sleeve-type-clamping mechanism 208 is used, FIG. 2B shows such a clamp in open, 220, and closed, 230, positions. A particular feature of one variation of clamp 200 in accordance with a specific embodiments of the present invention is the presence of complementary mortises 213 along the inner surfaces of clamp members 201 and 202 such that mortises 213 are specific for rivet-shaped implantable device 100, as shown in FIG. 2C. Top 102 of female rivet component 101 and top 103 of male component 109 are complementary to mortises 213 of clamp members 201 and 202, thereby facilitating the union of implantable device 100 for securing tissue within a graft. Note that in this particular embodiment of clamp 200 a wing-nut-type clamp closure 210, having a wing nut 212, a threaded shaft 214, and a bolt 216 is used as the clamp closure mechanism.

Similarly, FIG. 3C shows another variation of clamp 200 in accordance with another specific embodiment of the present invention having complementary mortises 215 along the inner surface of clamp members 201 and 202 specific for staple-shaped implantable device 110. Top 112 of female staple component 111 and top 113 of male staple component 119 are complementary to mortises 215 of clamp members 201 and 202. Upon closure of clamping members 201 and 202, union of implantable device components 111 and 119 occurs, thereby securing, for example, tendon or ligament replacement tissue 002 and/or 003 to bone core 001 in a graft such as that shown. Note that in this particular embodiment of clamp 200, sleeve-type clamp closure 208 is used as the clamp closure mechanism.

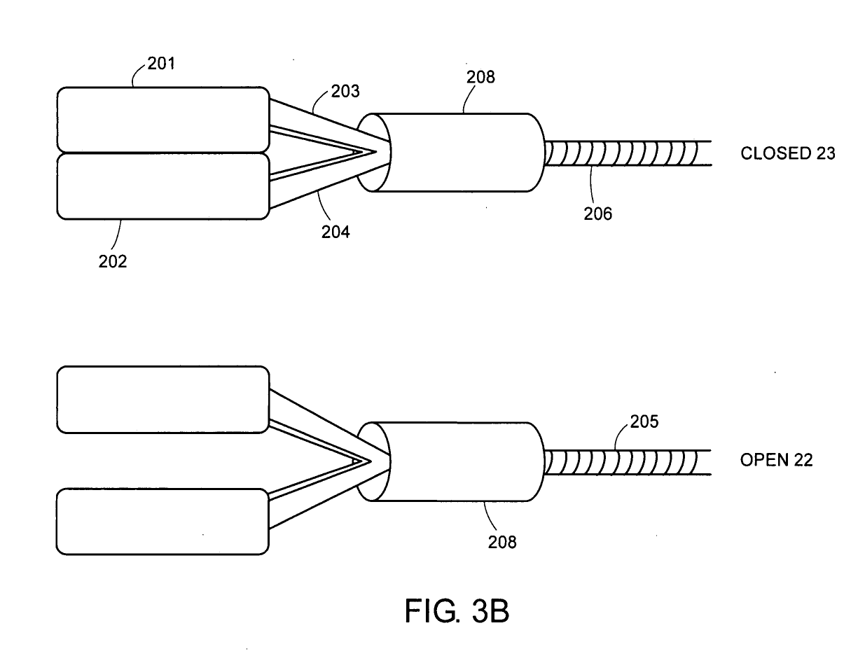

Several variations of a circlage-type clamp in accordance with specific embodiments of the present invention are shown in FIG. 4. FIG. 4A shows a basic circlage-type clamp 300 having two arms 302 and 304 pivotally connected at pivot joint 306, further connected to two arcuate clamping members 308 and 310. Arms 302 and 304 further have two circular handle grips 301 and 303 with a locking mechanism having corresponding toothed components 306 and 307 for locking and unlocking clamping members 308 and 310. Note that in this particular depiction of circlage-type clamp 300 a graft fastener 130 comprising two semi-circle halves 131 and 131′ enclosing a composite graft is shown within recess 309 formed between clamping members 308 and 310.

FIG. 3B depicts circlage-type clamp 300 enclosing graft fastener 120 having a circle configuration with a soft tissue graft within. A detail of arcuate clamping members 308 and 310 and recess 309 having no graft or graft fastener within recess 309 is shown in FIG. 4C. A particular feature of the circlage clamp of the specific embodiment as best shown in FIG. 4C is the presence of complementary mortises 311 having specificity for graft fasteners having a circle or semi-circle configuration.

Yet another variation of a circlage-type clamp can be seen in FIG. 4D wherein circlage clamp 350, having two arms 352 and 354 pivotally connected at pivot joint 356 are connected to two clamping members 358 and 360 which define an inner recess 359. At the distal end of arm 358 is a cannulated cylinder, parallel to the longitudinal axis of arm 388 and disposed along the recess-side of arm 358. Circlage clamp 350 further comprises an expandable and contractible loop 368 comprising a flexible band 367 having a first ringed end 364 and a second non-ringed end 366 defining an inside surface 363 and an outside surface 365 when non-ringed end 366 is threaded through ringed-end 364. First ringed end 364 is further rigidly connected to the recess-side of arm 360 and essentially perpendicular to the longitudinal axis of arm 360. Ringed end 364 is further disposed on the outer surface of flexible band 367, and rigidly connected and essentially perpendicular to the tangent T of flexible band 367 at connection point 370. Second non-ringed end 366 is further movably connected to the distal end of arm 358 via cannulated cylinder 362. Cannulated cylinder 362 further contains unidirectional teeth along the inner surface of the cylinder (not shown) to engage opposing unidirectional teeth 369 along outer surface 365. Using handles 351 and 352 to decrease recess 359, unidirectional teeth within cannulated cylinder 362 (not shown) engage unidirectional teeth 369 of loop 368. Loop 368 thus constricts around circle-shaped graft fastener 120, and thereby secures tissue in the composite graft located within graft fastener 120.

If the graft fastener, such as a circle-shaped graft fastener is too rigid and cannot be bent as desired, heat may be applied to increase the flexibility of the bioabsorbable material.

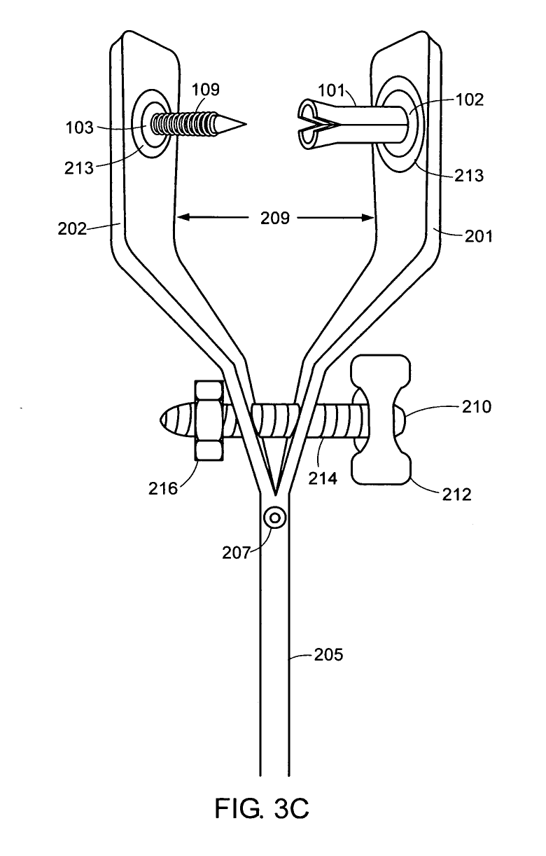

FIG. 5A shows a composite graft 400 of a type in accordance with the present invention. Composite graft 400 comprises a bone core 402, tendons 404, and an implantable device such as rivet-shaped graft fastener 100. As seen in FIG. 5B, soft tissue graft 410 can also be formed and secured with a graft fastener such as circle-shaped fastener 125 having interior spikes to prevent slippage 127, which is a variation of circle-shaped fastener 120 shown in FIG. 1C. Note that a circlage-type clamp such as example 300 (shown also in FIG. 3A) may also be used to close graft fastener 125.

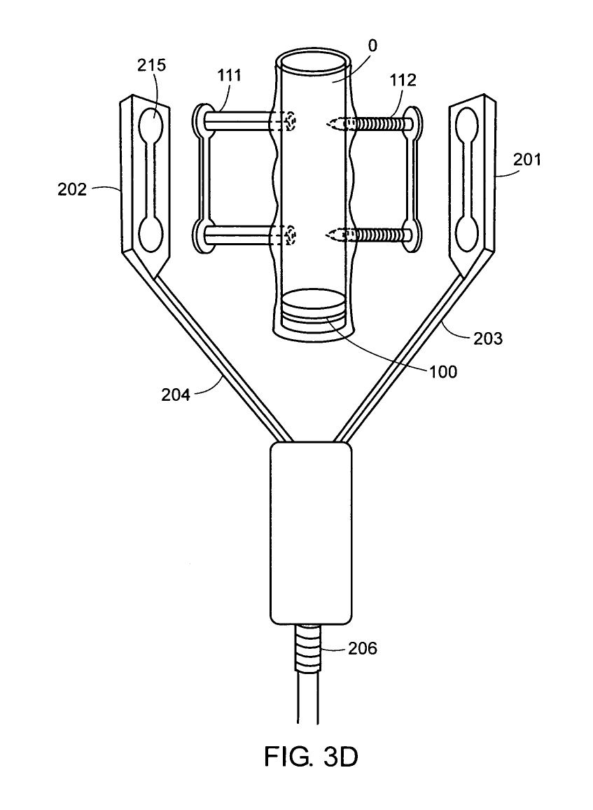

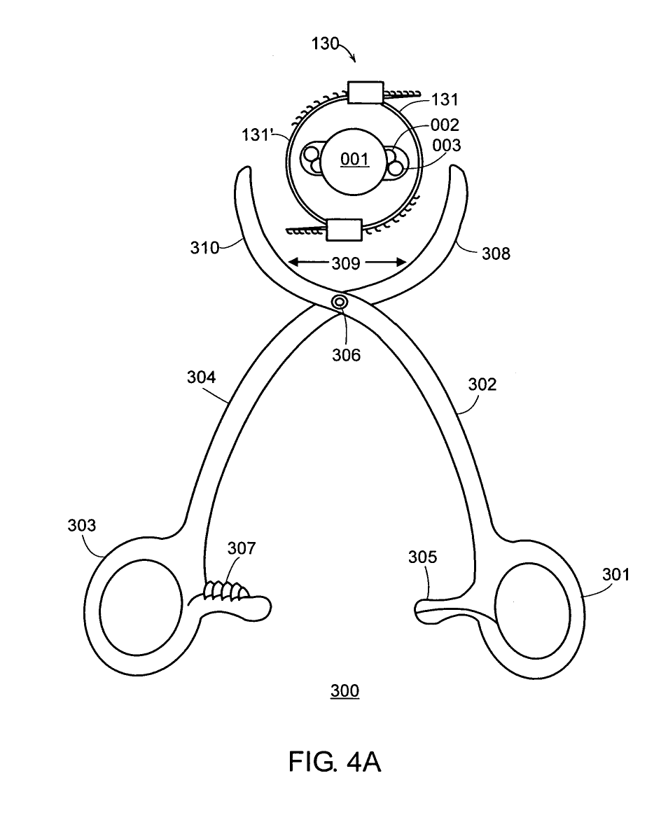

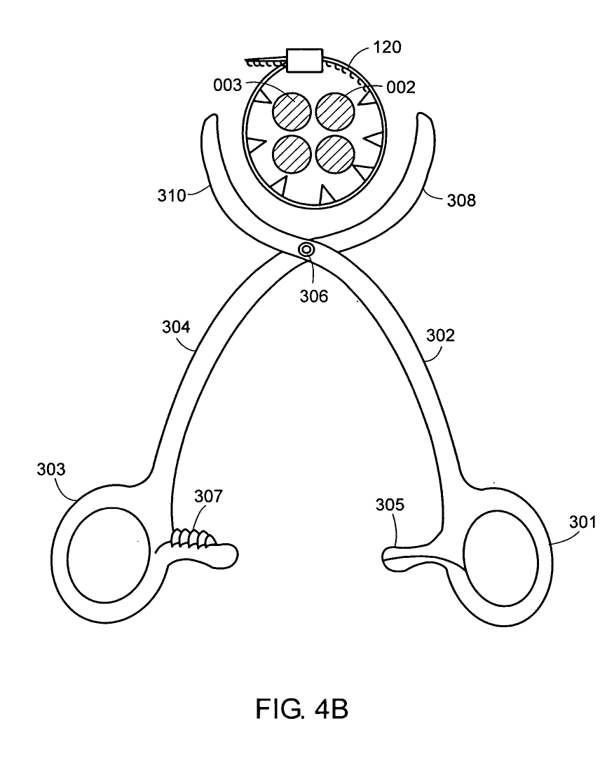

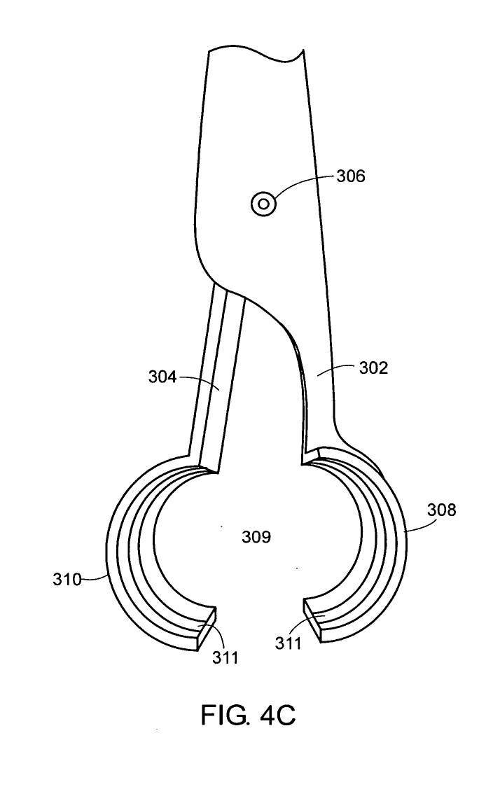

Referring to FIGS. 6A-6D, a bone plug 001, either from an allogenic or autogenous bone source, has two longitudinal substantially parallel grooves that are drilled on opposite sides of each bone plug. The grooves provide a recess in which the semitendenous tendon 001 and gracilis 003 can be placed. A notch 08 may also be drilled across one end of the bone plug so that the tendon can be wrapped alongside and around the end of the bone plug without protruding excessively. Notch 08 is optional, however, because the bone tunnel is open at each end. It is advantageous to provide implantable device holes through the bone plugs such as 07 (see FIG. 6B) for rivet-shaped implantable devices, or alternatively 09 (see FIG. 6D) for staple-shaped implantable devices. With such holes, the tendon can be readily secured to the bone plug, as depicted in FIGS. 6A and 6C (tendon not shown). Implantable device holes 07 and 09 are drilled radially through the bone plug and from one of the substantially parallel grooves 06 to the other.

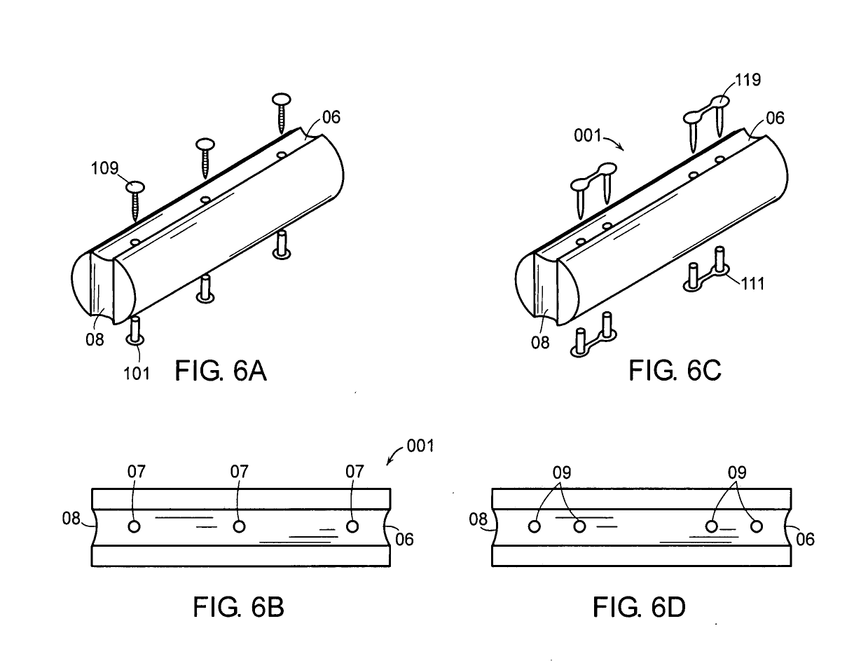

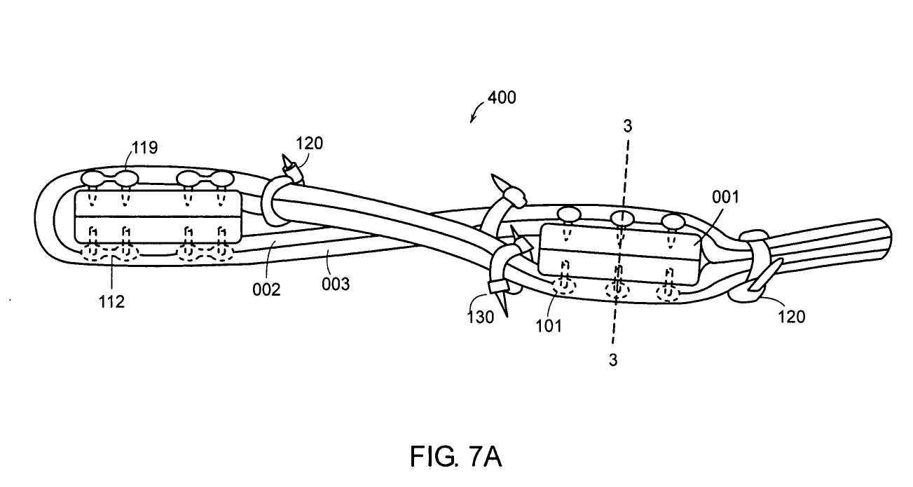

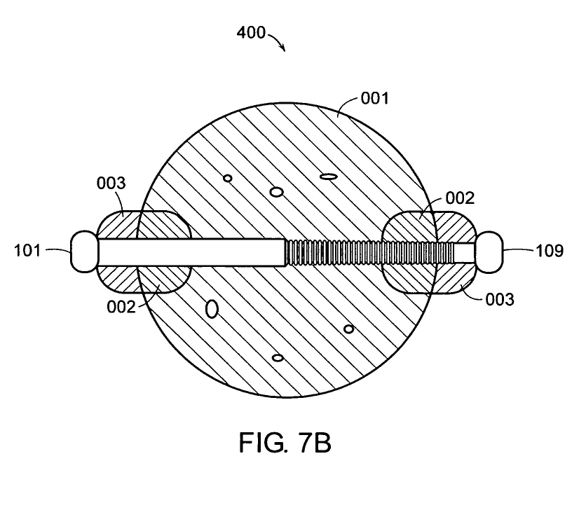

To form the essentially two parallel grooves 06 in bone plug 001 for making a composite graft 400, a bone block drill guide 900 in accordance with a preferred embodiment of the invention may be used, and is shown in FIGS. 8A and 8B. Drill guide 900 comprises a central substantially cylindrical column 902 having a pair of opposing curved walls 904 that have a center of curvature substantially coincident with the center axis through column 902. Curved walls 904 are shaped so as to hold a bone plug parallel with the axis of column 902 and substantially centered within. A second pair of opposing curved walls is situated 180° from the other with respect to central column 902 formed by curved walls 904. This second pair of opposing curved walls comprises the drill guide walls 906. Drill guide walls 906 forms two parallel columns on opposite sides of central column 902 and have a shorter radius of curvature than first pair of opposing walls 904. In accordance with a preferred embodiment, the inner diameter of drill guide walls 906 is 6 mm whereas the inner diameter of first opposing walls 904 is 11 mm. Central column 902 is mounted over a base 910 such that a bone plug situated in central column 902 rests on base 910. Base 910 is provided with holes there through in alignment with the open circular cylinder formed within drill guide walls 906. Base 910 may also include legs 908 for supporting drill guide 900 over a table. For drilling implantable device holes through the bone plug, holes 912 are arranged horizontally through drill guide walls 906 and spaced appropriately to accommodate either rivet-shaped or staple-shaped implantable devices. Three holes are preferably aligned in a line for rivet-shaped implantable devices, whereas two groups of two hole are preferable aligned in a line for staple-shaped implantable devices.

The substantially parallel grooves 06 are drilled by inserting bone plug 001 into the center chamber of column 902 formed by opposing curved walls 904. A drill is directed down column 902 along each of drill guide walls 906 in succession. Thus, parallel grooves 06 are formed on opposite sides of bone plug 001. The drill may be equipped with a stop to prevent the drill from being directed too far down through column 902 where it may contact the table beneath. A drill bit inserted through holes 912 can be easily directed through the center of groove 06 along the bone plug, thereby drilling implantable device hole 912 from one groove through to the opposite groove in the bone plug.

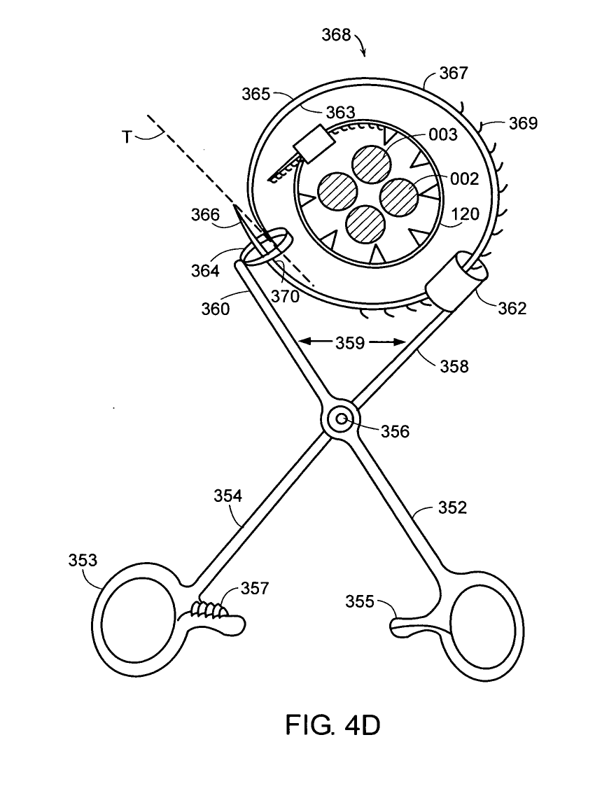

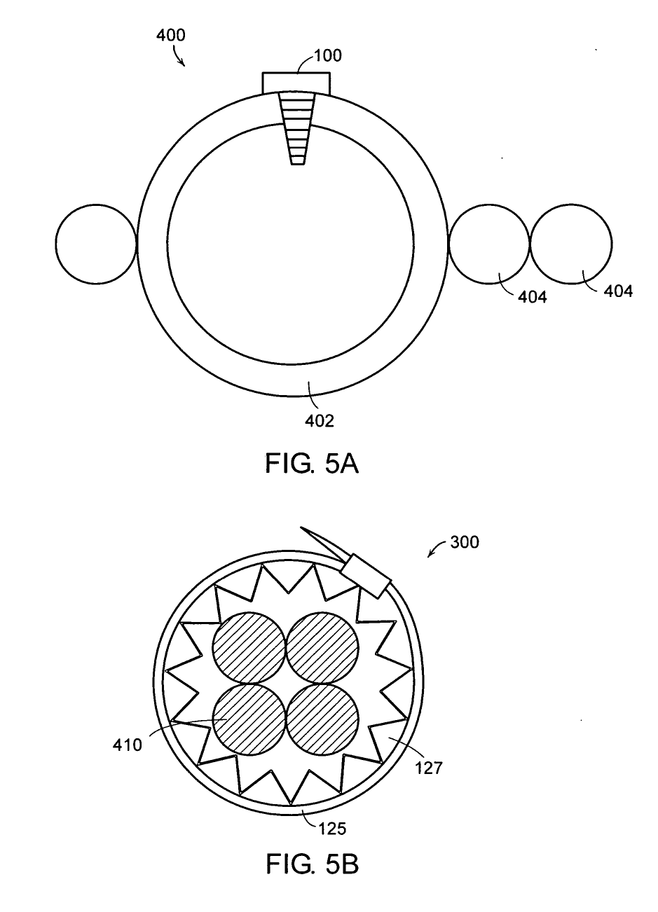

A semitendonous tendon 002 and/or gracilis 003 is extended between both of two bone plugs 001, as seen in FIG. 7A. Tendons 002 and/or gracilis 003 are seated inside the two substantially parallel grooves 06 and about an end of each bone plug. Tendons 002 and/or gracilis 003 are preferably secured to themselves using bioabsorbable implantable devices such as 120 and 130 to form a double loop as shown in FIG. 7A. Implantable devices such as 100 and 110 are also inserted through implantable device holes to attach tendon 002 to each bone plug 001. The tendon strands may be straight or twisted between bone plugs 001. Twisting will shorten the length of the graft. A ligament replacement may include both semitendonous tendon 002 and gracilis 003. As such, four strands will connect the two bone plugs. Other embodiments may use one or the other of semitendonous tendon 002 and gracilis 003. Still other embodiments may substitute or combine human-made or artificial fibers or human tissue for the tendon to be used as the ligament replacement. A cross-section of a completed composite graft can be seen in FIG. 6B, taken along the line 3-3 of FIG. 7A. Semitendonous tendons 002 and gracilis 003 are secured to bone plug 001 in this particular embodiment using rivet-shaped implantable device 100, but staple-shaped implantable device 110 can also be used or any other shape of implantable device that is capable of securely attached tendon to bone. Both rivet-shaped device 100 and staple-shaped device 110 are shown in FIG. 7A.

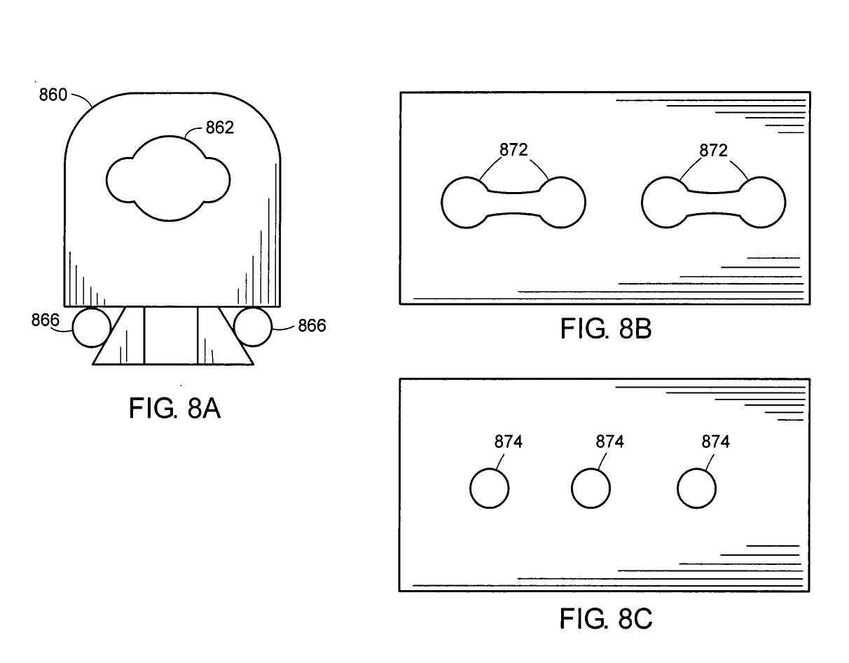

An alternative method of making composite graft 400 can be accomplished by use of a graft preparation table such as that of a preferred embodiment 800 shown in FIG. 9. Table 800 includes a series of holes 802 in which a bone-block drill guide such as column 902 having matching pegs along its bottom may be supported. FIG. 9 illustrates a bone-block drill guide 844 having drill guide walls 904 and 906 as described above with respect to drill guide 900. A drill guide cap 846 is provided with two holes 848 wherein each hole aligns with one of two parallel columns on opposite sides of central column through bone-block guide 844. Cap 846 and bone-block drill guide 845 has mating alignment pins and holes for guaranteeing correct alignment. Cap 846 may be chained to bone-block drill guide 844 to make sure it is not lost.

After inserting a bone plug into bone-block drill guide 844, cap 846 is placed into alignment over the guide. The longitudinal grooves are drilled by inserting the drill bit down through holes 848 in cap 846. After drilling the grooves, cap 846 is removed. The bone block can be removed by lifting bone-block drill guide 844 off graft preparation table 840 and pushing the bone block out of the guide. Graft preparation table 840 may be additionally provided with sizing tubes 850 of different diameters. The sizing tubes are useful in determining the diameter of a bone block. A linear scale 852 may also be included for permitting length measurements.

Attaching semitendonous tendon 002 and gracilis 003 in a loop about two bone blocks 001 completes composite graft 400. Implantable device blocks 860 and 861 assist in this process. An implantable device block 860 as shown in FIG. 8A includes a tunnel 862 that has two channels opposite one another across the central hole. The channels accommodate tendon 002 and gracilis 003 looped about bone block 001. Implantable device block 860 rides on a track 864 on graft preparation table 840. Two parallel rails 866 form track 864. Implantable device block 860 includes a pair of grooves for holding bone block 001 against rails 866 and permitting bone block 001 to ride along the rail. A ratchet rod 868 on implantable device block 861 is used to apply tension on the ligament replacement looped about bone blocks 001. Implantable device blocks 860 and 861 include a vertical hole for accepting a stop pin 869 for holding bone block 001 in place within its tunnel. Pin 869 prevents bone block 001 from being pulled out of implantable device block 860 when graft 400 is being tensioned.

Implantable device block 860 is fixed in position on the track and implantable device block 861 is fixed to ratchet rod 868. Ratchet rod 868 has a series of grooves spaced along its length and travels in a tube parallel to track 864. The tube includes ridges along its inner circumference that engage walls of the grooves on ratchet rod 868. The ridges and grooves prevent ratchet rod 868 from moving attached implantable device block 860 toward implantable device block 861. The tension on the semitendonous tendon and gracilis loop can be increased by pulling out the ratchet rod 868 to achieve the desired tension. To release the tension, ratchet rod 868 is rotated to disengage the grooves from the ridges. Ratchet rod 868 may then be pushed back into its tube.

As can be seen in FIGS. 8B and 8C, the sides of each implantable device block 860 include openings 872 or 874 to allow insertion of implantable devices for securing tendon or ligament replacement tissues 002 and/or 003 to bone block 001. Openings 872 are sufficiently large and spaced and aligned to accommodate staple-shaped implantable devices 110 whereas openings 874 are sufficiently large and spaced and aligned to accommodate rivet-shaped implantable devices such as 100. Each implantable device block contains the equally shaped and aligned openings on opposites of the block. Thus, female and male components of implantable devices such as 100 or 110 can be inserted from opposite sides of each block through the ligament replacement and bone block to secure the ligament replacement loops to the bone blocks.

Insertion of implantable devices such as 100 and 110 may be facilitated by use of clamps such as those shown in FIGS. 3A-3D. Once ligament replacement tissues 002 and/or 003 are secured to both bone blocks 001, the tension between implantable device blocks 860 and 861 can be released and composite graft 400 removed. Composite graft 400 is now ready for use in the surgical reconstruction.

While the above methods discuss the making of a composite graft such as 400 using autogenous or allogenic bone for the source of bone plug 001, it is envisioned that synthetic graft material could also be used for both the bone plug and ligament replacement tissues. In addition, it is envisioned that totally soft tissue grafts can be made in conjunction with specific embodiments of the present invention, particularly by using implantable devices such as 120 and 130 for securing the soft tissue together.

The present invention as expressed above may be embodied in other specific forms without departing from the true scope of the invention. The described embodiments are to be considered in all respects only as illustrative and not restrictive.

1. A bioabsorbable implantable device for replacing sutures in construction of a composite graft in ligament replacement surgery comprising a female component and a male component.

2. A bioabsorbable implantable device for replacing sutures in construction of a composite graft in ligament replacement surgery in the shape of a rivet.

3. The implantable device of claim 1 wherein the female component contains unidirectional teeth on an interior surface and the male component contains opposing unidirectional teeth on an external surface.

4. The implantable device of claim 1 wherein the components of the device are in the shape of a staple.

5. The implantable device of claim 4 wherein the female component contains unidirectional teeth on an internal surface and the male component contains opposing unidirectional teeth on an external surface.

6. The implantable device of claim 1 wherein the device forms a circle when the male component is inserted into the female component.

7. The implantable device of claim 6 wherein the female component of the device contains unidirectional teeth on an internal surface and the male component of the device contain opposing unidirectional teeth on an external surface.

8. The implantable device of claim 6 wherein an inner arc defined by the circle contains protruding spikes.

9. The implantable device of claim 6 wherein the device is in the shape of a semicircle with a portion of the male component extending beyond the semicircle.

10. The implantable device of claim 9 wherein the female component of the device contains unidirectional teeth on an internal surface and the male component of the device contain opposing unidirectional teeth on an external surface.

11. The implantable device of claim 9 wherein a first device is combined with a second device to complete a circle.

12. The implantable device of claim 9 wherein an inner arc defined by the semicircle contains protruding spikes.

13. The implantable device of claim 9 wherein the male component of a first device is inserted into the female component of a second device and the male component of the second device is inserted into the female component of the first device, to complete a circle.

14. The implantable device according to claim 1, wherein the bioabosrbable device contains a plurality of bioabsorbable materials.

15. The implantable device of claim 1 wherein the bioabsorbable device contains one of a group of compounds comprising polydextrolactic acid, polylevolactic acid, pologlycolites, and polydioxanoe.

16. The implantable device of claim 1 wherein the bioabsorbable material is polydextrolactic acid.

17. The implantable device of claim 1 wherein the bioabsorbable material is polylevolactic acid.

18. The implantable device of claim 1 wherein the bioabsorbable material is polydioxanoe

19. The implantable device of claim 1 wherein the bioabsorbable material is a polyglycolite.

20. A securing device for securing graft tissue in a graft using a bioabsorbable implantable device according to claim 1, comprising:
A. first and second arms pivotally connected to one another, each arm having a distal and a proximal end that define a recess, the distal end of the second arm further comprising a cannulated cylinder wherein the cannulated cylinder is attached along the recess side of the second arm such that the cylinder is parallel to the longitudinal axis of the second arm, the cannulated cylinder further having unidirectional teeth along the inner surface of the cylinder;
B. an expandable and contractible loop connected to the first and second arms comprising;
i. a linear flexible band having a first ringed end and a second non-ringed end and an inside and an outside surface comprising;
a. unidirectional teeth on the outside surface of the second non-ringed end;
b. a rigid connection between the first ringed end and the outer surface of the flexible band such that the plane of the ring is essentially perpendicular to the tangent of the flexible band at the connection point;
c. a lariat-like conformation such that the second non-ringed end is threaded through the first ringed end such that the inside surface of the second end is juxtaposed to the outside surface of the first end within the ring;
ii. a fixed connection between the first ringed end of the circular loop and the distal end of the first arm such that the first ringed end is connected to the recess-side of the first arm and essentially perpendicular to the longitudinal axis of the first arm.
iii. a movable connection between the non-ringed end of the circular loop and the distal end of the second arm wherein the non-ringed end of the circular loop is threaded through the cannulated cylinder at the distal end of the second arm such that the unidirectional teeth on the outside surface of the non-ringed end of the circular loop are capable of being engaged by the opposing unidirectional teeth within the cannulated cylinder at the distal end of the second arm as the recess between the two arms is decreased; and
iv. a mechanism for expanding the dimension of the circular loop after it has been contracted.

21. In a surgical clamp for use in securing tissue of the type wherein the clamp comprises a pair of opposing clamping members for engaging tissue; and of the type wherein a first arm is attached at its distal end to one clamping member and a second arm is attached at its distal end to the opposite clamping member; and of the type wherein the first and second arms are pivotably connected to one another for variably adjusting a distance between the respective clamping members, the improvement comprising:
clamping members having mortises complementary to bioabsorbable graft fastener shapes along the inner surface of the clamping members.

22. A surgical clamp for use in securing tissue according to claim 21 wherein each of the clamping members is arcuate in shape.

23. A surgical clamp for use in securing tissue according to claim 21 wherein the clamping members are parallel to each other.

24. A surgical clamp for use in securing tissue according to claim 22 wherein the mortises are complementary to a circlage-type graft fastener.

25. A surgical clamp for use in securing tissue according to claim 23 wherein the mortises are complementary to a rivet-shaped graft fastener.

26. A surgical clamp for use in securing tissue according to claim 23 wherein the mortises are complementary to a stapler-shaped graft fastener.

27. A surgical clamp for use in securing tissue according to claim 21, further including a locking mechanism for securely maintaining an engagement between the clamping members.

28. A surgical clamp for securing tissue according to claim 22 wherein one clamping member is sized to partially receive the other clamping member.

29. An implantable device insertion station comprising:
a tensioning track;
a pair of blocks for inserting bioabsorbable implantable devices, separated along said tensioning track, each block having a chamber for accommodating a bone block and openings for bioabsorbable implantable devices on opposite sides of the block sufficiently large to expose the tendons and accommodate the implantable devices, the implantable device openings of the opposite sides being aligned with one another with the chamber therebetween;
restraints for holding the implantable device blocks an adjustable distance apart from one another along said tensioning track.

30. The implantable device insertion station of claim 29 wherein the chamber in each implantable device block has a central tunnel and first and second parallel tunnels intersecting opposite sides of the central tunnel and being parallel to the central tunnel.

31. The implantable device insertion station of claim 29 wherein the implantable device openings of each implantable device block are aligned so that a bioabsorbable implantable device inserted into the implantable device block through one of the implantable device openings can be extended through each of the central tunnel, the first tunnel and second tunnel, and into the implantable device opening on the opposite side.

32. The implantable device insertion station of claim 29 wherein said means for holding comprises a ratchet rod having a series of grooves for adjustable engaging ridges that are fixed in position relative to said tensioning track.

33. The implantable device insertion station of claim 32 wherein the ratchet rod is arranged with respect to the ridges so that axially rotating the ratchet rod can disengage the grooves from the ridges.

34. The implantable device insertion station of claim 29 wherein each implantable device block includes an orifice for receiving a stop pin for holding a bone block within the chamber.

35. An improved graft preparation table of the type in which a body, mounted on a base, has a central tunnel for receiving a bone block with first and second parallel tunnels intersecting opposite sides of the central tunnel and being parallel to the central tunnel, and in which a tensioning track is mounted on the base, wherein the improvement comprises:
a pair of blocks for inserting bioabsorbable implantable devices, separated along said tensioning track, each block having a chamber for accommodating a bone block and openings for bioabsorbable implantable devices on opposite sides of the block sufficiently large to expose the tendons and accommodate the implantable devices, the implantable device openings of the opposite sides being aligned with one another with the chamber therebetween; and
restraints for holding the implantable device blocks an adjustable distance apart from one another along said tensioning track.

36. The improved graft preparation table of claim 35 wherein said means for holding comprises a ratchet rod having a series of grooves for adjustably engaging ridges that are fixed in position.

37. The improved graft preparation table of claim 35 further comprising a plurality of sizing tunnels mounted on said base.

38. An instrument for perforating a tendon/bone complex of a prepared composite graft which comprises:
a drilling component;
means for immobilizing the composite graft with the two parallel opposite tendon/bone complexes exposed;
means for guiding the placement of the drill along the exposed tendon/bone complex of the composite graft;
means for producing a plurality of channels of desirable shape from one side of the graft directly through to the opposite side of the graft to allow insertion of compatibly-shaped bioabsorbable implantable devices.

39. In a method of making a graft, of the type wherein a bone block is inserted into a bone block drill guide, said drill guide having a central column supported by a base, and first and second parallel cylindrical columns intersecting opposite sides of the central column equidistant from the center of the central column, said first and second cylindrical columns each having a center, the centers of the central column and the first and second columns all being aligned; and of the type wherein the bone block inserted in the drill guide is drilled to create first and second parallel grooves in opposite sides of the bone block; and of the type wherein a ligament replacement is attached to the bone block and looped about the bone block along the first and second grooves; and of the type wherein the ligament replacement is tensioned appropriately, the improvement comprising:
attaching the ligament replacement while tensioned to the bone block using a bioabsorbable implantable device.

40. In a method of making a graft, of the type wherein first and second bone plugs, each having on its outer surface two longitudinal substantially parallel grooves opposite one another on said each bone plug; and of the type wherein at least one ligament replacement is extended between both of the first and second bone plugs along two substantially parallel grooves in each bone plug; and of the type wherein the first and second bone plugs are held apart from one another to tension the at least one ligament replacement, the improvement comprising:
attaching the at least one ligament replacement to the first and second bone plugs while they are being held apart by means of a bioabsorbable implantable device.

41. The method of claim 40 further comprising inserting the first and second bone plugs along with the at least one ligament replacement each into an implantable device insertion block before said step of holding, accommodating one of the bone plugs and the at least one ligament replacement in a compatible chamber on each implantable device insertion block; and aligning implantable device openings on opposite sides of one another with the chamber therebetween.

42. The method of claim 41 further comprising inserting a stop pin into each of the implantable device insertion blocks before the step of holding so as to keep each bone plug within its respective implantable device insertion block.

43. The method of claim 40 wherein the said step of attaching a bioabsorbable implantable device includes applying simultaneous pressure to opposite sides of the bone plugs at the exposed surfaces of the opposing bioabsorbable implantable device components, thereby locking the ratchet teeth of each component together to achieve the desired clamping force for the implantable device and securing the at least one ligament replacement to the first and second bone plug.

44. The method of claim 39 further comprising using a bioabsorbable implantable device in the shape of a rivet.

45. The method of claim 39 further comprising using a bioabsorbable implantable device in the shape of a staple.

46. The method of claim 39 further comprising using a bioabsorbable implantable device that completes a circle when the male component is inserted into the female component.

47. The method of claim 39 further comprising using a bioabsorbable implantable device in the shape of a semicircle with an end extending beyond the semicircle arc.

48. The method of claim 46 further comprising using a bioabsorbable implantable device wherein the inner arc of the completed circle contains protruding spikes.

49. The method of claim 47 further comprising using a bioabsorbable implantable device wherein the inner arc defined by the semicircle contains protruding spikes.

50. The method of claim 40 further comprising securing the bioabsorbable implantable device to the at least one ligament replacement and corresponding bone plug using a constricting cinch-type clamp.

51. The method of claim 40 further comprising securing the bioabsorbable implantable device to the at least one ligament replacement and corresponding bone plug using a circlage clamp.

52. The method of claim 40 further comprising securing the bioabsorbable implantable device to the at least one ligament replacement and corresponding bone plug using a parallel vice-like clamp.

53. A method of making a graft according to claim 40, further comprising securing an autogenous bone block to the at least one ligament replacement tissue wherein the at least one ligament replacement tissue is selected from the group including autogenous and allogenic tissue.

54. A method of making a graft according to claim 40, further comprising securing an allogenic bone block to the at least one ligament replacement tissue wherein the at least one ligament replacement tissue is selected from the group including autogenous and allogenic tissue.

55. A method of preparing a soft tissue graft using a bioabsorbable implantable device according to claim 39 comprising securing a first soft tissue to a second soft tissue wherein the each of first and second soft tissue is selected from the group including autogenous and allogenic tissue.

56. A method of making a soft tissue graft using a bioabsorbable implantable device according to claim 39 comprising securing an allogenic first soft tissue to an allogenic second soft tissue wherein the soft tissue graft is prepared prior to surgery as a pre-fabricated graft.

57. A method of making a graft using a bioabsorbable device according to claim 39 comprising securing an allogenic bone plug to an allogenic soft tissue wherein the graft is prepared outside the operating room as a pre-fabricated graft.

58. A method of making a graft using a bioabsorbable implantable device according to claim 1 comprising securing a bone plug to an allogenic tissue wherein the graft is prepared outside the operating room as a pre-fabricated graft.

59. A package for use in reconstructive surgery comprising:
a sterile container;
a graft which is of a predetermined length and a predetermined width; and
a bioabsorbable implantable device.

60. A package according to claim 59, wherein the graft is formed from an allogenic graft.

61. A package according to claim 59, wherein the graft is formed from man-made fibers.

62. A package according to claim 59, wherein the graft resides in sterilized fluid within the sterile container.

63. A package according to claim 62, wherein the sterile container is hermetically sealed

64. A package according to claim 59, wherein the reconstructive surgery is knee surgery.

65. A package according to claim 59, wherein the package further includes:
a pair of bone blocks.

66. A package according to claim 59, wherein the container contains markings as to graft size.

67. A package according to claim 66, wherein the markings indicate graft width and graft length.

68. According to claim 59, wherein the graft has markings at points for attachment to bone blocks.

69. A graft for use in reconstructive surgery comprising:
a tendon;
a pair of bone blocks; and
at least one bioabsorbable fastener coupling the tendon and the pair of bone blocks.

70. A package according to claim 69, wherein each bone block has a cavity through a central axis into which the tendon is placed.

71. A package according to claim 70, wherein each bone blocks is provided with at least one groove into which the tendon sits.

72. A package according to claim 71, wherein each bone block is part of the package.
